(19) United States
(12) Patent Application Publication
(10) Pub. No.: US 2023/0310792 A1
(43) Pub. Date: Oct. 5, 2023

(54) SYSTEMS AND METHODOLOGIES FOR PERFORMING BRAINWAVE ENTRAINMENT USING NESTED WAVEFORMS

(71) Applicant: Reversal Solutions, Inc., Belmont, CA (US)

(72) Inventors: John A Fortkort, Austin, TX (US); PETER A. NEWSOM, Woodside, CA (US); Annelise E. Barron, Redwood City, CA (US)

(21) Appl. No.: 18/017,420

(22) PCT Filed: Jul. 22, 2021
(86) PCT No.: PCT/US2021/042675

§ 371 Date: Jan. 23, 2023

Related U.S. Application Data
(60) Provisional application No. 63/055,320, filed on Jul. 22, 2020.

(51) Int. Cl. A61M 21/00; G06F 3/01; G16H 20/70

(52) U.S. Cl. CPC A61M 21/00; G06F 3/015; G16H 20/70; A61M 2021/0027; A61M 2021/0044

(57) ABSTRACT

Systems and methodologies are provided for performing brainwave entrainment on a subject. An exemplary embodiment of these methodologies includes providing a signal source selected from the group consisting of audio signal sources and video signal sources; and using the signal source to apply to the subject, or to induce the formation of in the subject, at least one nested waveform selected from the group consisting of audio waves and electromagnetic waves.

### CROSS-REFERENCE TO RELATED APPLICATION

The present application is a national stage filing of PCT/US21/42675, filed on Jul. 22, 2021, which has the same title and the same inventors, and which is incorporated herein by reference in its entirety; which claims the benefit of priority from U.S. Provisional Application Mo. 63/055,320, filed Jul. 22, 2020, entitled “SYSTEMS AND METHODOLOGIES FOR TREATING OR PREVENTING PSYCHIATRIC DISORDERS WITH BRAIN ENTRAINMENT USING NESTED FREQUENCIES”, which has the same inventors, and which is incorporated herein by reference in its entirety.

### FIELD OF THE DISCLOSURE

The present application relates generally to light therapy, and more specifically to methods for performing brainwave entrainment utilizing nested waveforms.

### BACKGROUND OF THE DISCLOSURE

Neurons in the human body use action potentials (APs) to transmit information. These brief and uniform pulses of electrical activity are generated when the membrane potential of a neuron reaches a threshold value. The resulting pulses travel down the axon toward synapses and terminate at postsynaptic neurons, where they initiate postsynaptic currents (PSCs). The PSCs then summate to either trigger or inhibit new APs. The resulting sequence or “train” of APs may contain information based on various coding schemes and may produce various results. In simple motor functions (such as, for example, muscle flexure), the strength at which these functions occur may depend solely on the firing rate of neurons. Other functions may rely on more complex temporal codes that are a function of the precise timing of single APs. These complex temporal codes may be tied to external stimuli (for example, those generated by the auditory system) or may be generated intrinsically by neural circuitry.

The human brain contains a large number of neurons. The electrochemical activity of neurons in generating the electrical currents required for APs occurs in a synchronized manner characterized by macroscopic oscillations. These oscillations may be described by their frequency, amplitude and phase, and may be monitored and depicted graphically in an electroencephalogram (EEG). The graphical depiction of these macroscopic oscillations in an EEG are often referred to as “brainwaves”.

Five common brainwave bandwidths (delta, theta, alpha, beta and gamma) have been identified in humans, each of which is associated with specific mental states. [Thompson, M., & Thompson, L. (2003). The neurofeedback book: An introduction to basic concepts in applied psychophysiology. Wheat Ridge, CO: The Association for Applied Psychophysiology and Biofeedback. Walter, V. J., & Walter, W. Grey]. These bandwidths are depicted in FIG. 1. Within these bandwidths, various sub-categories (high, low alpha and beta, and sensorimotor rhythm) have also been identified, which are associated with different mental activities.

By way of example, delta waves (0.5-3 Hz) are the dominant brainwaves observed during deep sleep. Theta waves (4-7 Hz) are typically associated with drowsy or relaxed states. Low alpha waves (8-10 Hz) are frequently associated with meditative states and inward thinking (for example, daydreams or dissociation from external stimulation). High alpha waves (11-12 Hz) are associated with creativity and the alert (but calm) state needed for peak performance. Sensorimotor rhythms (13-15 Hz), which are frequently categorized as low beta, are believed to occur predominantly in the still state before a reactive psychomotor action. Low beta waves (16-20 Hz) are associated with intellectual activity and problem-solving. High beta waves (21-37 Hz) are found in emotional and anxious states. Gamma waves (38-42 Hz) are associated with attention and intense cognitive activity. [Id.] An excess of brainwave activity in any of the foregoing bandwidths or sub-categories may also be associated with a particular state or condition. Thus, for example, excessive beta and gamma activity has been associated with hyper-aroused states, such as those occurring during stress, anxiety, or insomnia. [Perlis, M. L., Merica, H., Smith, M. T. & Giles, D. E. (2001). Beta EEG activity and insomnia. Sleep Medicine Reviews, 5(5), 363-374].

Brainwave entrainment (sometimes referred to as brainwave synchronization or neural entrainment) may be utilized to modulate the brainwaves in a subject to induce, for example, a particular mental state in the subject. Brainwave entrainment typically involves the manipulation of the frequency of brainwaves (or the associated patterns of firing of neural synapses) by suitable rhythmic or periodic external stimuli, which may include auditory, visual, or tactile stimuli. The effectiveness of brainwave entrainment is believed to result from the tendency of the brain to naturally synchronize its brainwave frequencies with the oscillations of periodic external stimuli. Since (as noted above) particular patterns of neural firing have been associated with certain mental states, it is believed that brainwave entrainment may be utilized to induce desired states of consciousness by modulating brainwaves in a subject. Such states of consciousness may be those which are conducive, for example, to studying, sleeping, exercising, meditating, or doing creative work.

Early work in brainwave entrainment focused on the use of visual stimuli. However, Chatrian et al. found that brainwave entrainment could also be achieved with auditory stimuli alone (specifically, clicking sounds). [Chatrian, E. G., Peterson, M. C., & Lazarte, J. A. (1960). Responses to clicks from the human brain: Some depth electrograph observation. Electroencephalography and Clinical Neurophysiology, 12, 479-489]. This led to the discovery by Oster that binaural beats (which are produced by the simultaneous application of first and second distinct, single frequency sine wave tones to first and second ears of a subject) stimulate brain activity that corresponds to the rhythm of the difference in the two stimuli frequencies. [Oster, G. (1973). Auditory beats in the brain. Scientific American, 229, 94-102].

It has since been found that the foregoing modes of brainwave entrainment (namely, visual and auditory entrainment) may be combined. This technique, which is the subject of U.S. 3,838,417 (Charas), is referred to variously as “audio visual stimulation” (AVS), “light and sound stimulation, ” “audio photic stimulation,” or “audio visual entrainment.” AVS has been utilized in various clinical applications involving attention deficit disorder (ADD), academic performance, cognition, depression, stress management, tension, pain, PTSD, migraine headaches, hypertension, and stroke.

AVS brainwave entrainment may be open-loop or closed-loop. In closed-loop AVS brainwave entrainment, the subject is attached to EEG recording electrodes. Brainwave activity is measured through these electrodes and is used by the AVS device to provide light and sound stimulation based on the properties of the brainwave activity recorded. Hence, the stimulation is driven by the subject’s brainwaves, and thus provides real-time feedback based on the neural activity of the user. This approach is termed “neurofeedback” and is often implemented with the assistance of a clinician.

By contrast, open-loop AVS brainwave entrainment is not dependent on the subject’s brainwave activity. In this approach, entrainment occurs in response to flickering light and audio tones of particular frequencies. Unlike the closed-loop approach, this form of AVS entrains brain activity in response to the designated frequencies (which are typically selected to induce a desired mental state), without any brain activity feedback provided to the AVS device. Various consumer products have been developed to implement open-looped AVS brainwave entrainment. These include, for example, the brainwave entrainment device sold under the trademark EquiSync®.

Other types of light therapy in particular have also been developed in the art that do not necessarily involve brainwave entrainment. For example, photobiomodulation therapy (PBMT) is a type of light therapy that utilizes non-ionizing electromagnetic energy to trigger photochemical changes in cellular structures that are receptive to photons. Various devices have been developed in the art to implement PBMT or processes related thereto. Examples of such devices are described, for example, in U.S. 2019/0246463A1 (Williams et al.)., U.S. US2019/0175936 (Gretz et al.), WO2019/053625 (Lim), U.S. U.S. 2014/0243933 (Ginggen), U.S. 2019/0142636 (Tedford et al.), U.S.7,354,432 (Eells et al.), U.S. 2008/0091249 (Wang), U.S. 10,391,330 (Bourke et al.) and U.S. 2016/0129278 (Mayer). Various salutary effects have been ascribed to PBMT including, for example, promotion of tissue healing or regeneration, reduction in inflammation, and general analgesic effects.

### BRIEF DESCRIPTION OF THE DRAWINGS

FIG. 1 is a graphical depiction of brainwaves from different frequency ranges. FIG. 1(a) depicts brainwaves from the delta band. FIG. 1(a) depicts brainwaves from the delta band. FIG. 1(b) depicts brainwaves from the theta band. FIG. 1(c) depicts brainwaves from the alpha band. FIG. 1(d) depicts brainwaves from the mu-rhythm band. FIG. 1(e) depicts brainwaves from the beta band. FIG. 1(f) depicts brainwaves from the gamma band.

FIG. 2 depicts nested waveforms generated by combining a theta signal at 5 Hz with a gamma signal at 30 Hz (FIG. 2A), 38.5 Hz (FIG. 2B) and 50 Hz (FIG. 2C).

FIG. 3 depicts a series of nested waveforms formed by combining a theta signal at 5 Hz with a gamma signal at 30 Hz (FIG. 3A), 35 Hz (FIG. 3B), 40 Hz (FIG. 3C), 45 Hz (FIG. 3D) and 50 Hz (FIG. 3E).

FIGS. 4-6 depicts a series of nested waveforms formed by combining a theta signal at 5 Hz with a gamma signal at 30 Hz (FIG. 4), 40 Hz (FIG. 5) and 50 Hz (FIG. 6).

FIG. 7 is a nested waveform made by combining theta signals of 5 Hz and 5.2 Hz to form pulses of about 30 Hz.

FIG. 8 is a nested waveform made by combining theta signals of 5 Hz and 7.4 Hz to create a waveform having multiple periodic amplitude modulations.

FIG. 9 is a nested waveform made by combining theta signals of 5 Hz and 9.8 Hz to create a waveform having an amplitude modulation of about 30 Hz.

FIG. 10 is a nested waveform made by combining theta signals of 5 Hz and 9.2 Hz to create a waveform having an amplitude modulation of about 10 Hz.

FIG. 11 is a nested waveform made by combining theta signals of 5 Hz and 9.6 Hz to create a waveform having an amplitude modulation of about 10 Hz.

FIG. 12 is a nested waveform made by combining theta signals of 5 Hz and 14.7 Hz to create a waveform having multiple periodic amplitude modulations.

FIG. 13 is a nested waveform made by combining theta signals of 8 Hz and 31.4 Hz to create a waveform having multiple periodic amplitude modulations.

FIG. 14 is a nested waveform made by combining theta signals of 8 Hz and 40.2 Hz to create a waveform having multiple periodic amplitude modulations.

FIG. 15 is a nested waveform made by combining theta signals of 8 Hz and 41.1 Hz to create a waveform having multiple periodic amplitude modulations.

FIG. 16 is a nested waveform made by combining theta signals of 8 Hz and 47 Hz to create a waveform having multiple periodic amplitude modulations.

FIG. 17 is an illustration of a gamma signal which has been modified with a theta signal (FIG. 17A) to obtain a nested waveform (FIG. 17B) in which the amplitude of the theta signal is modified at the frequency of the gamma signal.

FIG. 18 is an example of a waveform in which only the rising portion of the theta signal is modified with a gamma signal.

FIGS. 19-22 are illustrations of an embodiment of a device which may be utilized to implement the brainwave entrainment methodologies disclosed herein.

### SUMMARY OF THE DISCLOSURE

In one aspect, a method is provided for performing brainwave entrainment in a subject. The method comprises (a) providing a signal source selected from the group consisting of audio signal sources and video signal sources; and (b) using the signal source to apply to the subject, or to induce the formation of in the subject, at least one nested waveform selected from the group consisting of audio waves and electromagnetic waves.

In another aspect, a method for treating an individual who has, or is at risk of developing, a psychological disorder. The method comprises (a) determining that an individual has, or is at risk of developing, a psychological disorder; and (b) performing brainwave entrainment on the individual using at least one nested waveform selected from the group consisting of audio waves and electromagnetic waves.

### DETAILED DESCRIPTION

While several open-loop brainwave entrainment devices and methodologies have been developed to date, further improvement is needed in these devices. For example, typical existing open-loop AVS brainwave entrainment devices utilize an entrainment signal at a single frequency which, in some cases, may be varied over time. This approach may be utilized, for example, to gradually bring a subject into a state of restfulness characterized by inducing greater theta wave activity in the brain. This may be accomplished, for example, by beginning the entrainment process using a higher frequency signal, and then gradually lowering the frequency of the signal to within the theta range.

However, as previously noted, the human brain utilizes brainwaves whose frequencies fall within at least five common brainwave bandwidths (delta, theta, alpha, beta and gamma), each of which is associated with specific mental states. Hence, using single frequency entrainment may limit the technique to addressing only one of these bandwidths at a time.

Moreover, brainwaves commonly occur in more than one of these frequency bandwidths concurrently. For example, the hippocampus supports not only long term memory encoding, but also plays a role in working memory maintenance of multiple items. While the neural mechanism underlying multi-item maintenance is not fully understood, theoretical work suggests that multiple items are maintained by neural assemblies synchronized in the gamma frequency range (25-100 Hz) that are locked to oscillatory activity (and in particular, to consecutive phase ranges of the oscillatory activity) in the theta frequency range (4-8 Hz). Indeed, cross-frequency coupling of the amplitude of high-frequency activity to the phase of slower oscillations has been found in both animals and in humans. Recent research suggests that simultaneous maintenance of multiple items in working memory is accompanied by cross-frequency coupling of oscillatory activity in the hippocampus, which is recruited during multi-item working memory. Moreover, maintenance of an increasing number of items is found to be associated with modulation of beta/gamma frequencies and amplitudes onto the theta band brain activity in both frequency and amplitude of this lower frequency. This is consistent with the hypothesis that longer cycles are required for an increased number of representations by gamma cycles. Research also suggests that the precision of cross-frequency coupling predicts individual working memory performance. The foregoing supports the hypothesis that working memory in humans depends on a neural code using phase information. [See Axmacher N, Henseler MM, Jensen O, Weinreich I, Elger CE, Fell J. Cross-frequency coupling supports multi-item working memory in the human hippocampus. Proc Natl Acad Sci U S A. 2010;107(7):3228-3233].

Other work in the field supports the thesis that various functions of the brain are dependent on cross-frequency coupling of brainwaves from different frequency domains. For example, robust coupling has been observed between the high- and low-frequency bands of ongoing electrical activity in the human brain. In particular, the phase of the low-frequency theta (4 to 8 hertz) rhythm modulates power in the high gamma (80 to 150 hertz) band of the electrocorticogram, with stronger modulation occurring at higher theta amplitudes. Furthermore, different behavioral tasks evoke distinct patterns of theta/high gamma coupling across the cortex. The results indicate that transient coupling between low- and high-frequency brain rhythms coordinates activity in distributed cortical areas, providing a mechanism for effective communication during cognitive processing in humans. [Canolty RT, Edwards E, Dalal SS, et al. High gamma power is phase-locked to theta oscillations in human neocortex. Science. 2006;313(5793):1626-1628].

Other work has elicited the nature of specific types of cross-frequency coupling. For example, a considerable amount of work has focused on phase-amplitude coupling (PAC), a form of cross-frequency coupling where the amplitude of a high frequency signal is modulated by the phase of low frequency oscillations. [Munia, T.T.K., Aviyente, S. Time-Frequency Based Phase-Amplitude Coupling Measure For Neuronal Oscillations. Sci Rep 9, 12441 (2019)].

It has been suggested that PAC is responsible for integration across populations of neurons, with lower frequency brain activity controlling the information exchange between brain regions by modulating the amplitude of higher frequency oscillations. In particular, spatially distributed coherent oscillations are thought to provide temporal windows of excitability that allow for interactions between distinct neuronal groups. It has been hypothesized that this mechanism for neuronal communication is realized by bursts of high-frequency oscillations that are phase-coupled to a low frequency spatially distributed coupling oscillation. This mechanism requires multiple physiologically different interacting sources (one generating the low-frequency coupling oscillation and the others generating phase-coupled high-frequency oscillations).

Support for the foregoing theory has been obtained using human intracranial EEG (iEEG) data, which provides evidence for multiple oscillatory patterns, as characterized on the basis of their spatial maps (topographies) and their frequency spectra. Indeed, the spatial maps for the coupling oscillations are found to be much more widespread than the ones for the associated phase-coupled bursts. Moreover, in the majority of the patterns of phase-amplitude coupling (PAC), phase-coupled bursts of high-frequency activity are synchronized across brain areas. In addition, working memory operations have been observed to affect the PAC strength in a heterogeneous way. In particular, working memory operations are found to increase the strength of some PAC patterns, while in others, working memory decreases it in the form of cross-frequency coupling where the amplitude of a high frequency signal is modulated by the phase of low frequency oscillations. [Maris, E., van Vugt, M., & Kahana, M. (2011). Spatially distributed patterns of oscillatory coupling between high-frequency amplitudes and low-frequency phases in human iEEG. Neuroimage, 54(2), 836-850].

In light of the foregoing, it will be appreciated that, while oscillatory brain activity reflects different internal brain states that may be characterized by the excitatory state of neurons and the synchrony among neurons, characterizing these states is complicated by the fact that different oscillations are often coupled (such as, for example, gamma oscillations nested in theta in the hippocampus). Moreover, changes in such coupling may reflect distinct mental states which may be characterized by oscillatory cycles based on distinct frequency and phase coupling. Consequently, single frequency brainwave entrainment may be insufficient or suboptimal in addressing these states, and its use may ignore potential advantages that may be attendant to entrainment in multiple regions simultaneously.

The foregoing issues may be addressed with the devices and methodologies disclosed herein. In one particular, non-limiting embodiment, these devices and methodologies feature the use in brainwave entrainment (and preferably, in open-loop AVS brainwave entrainment) of a nested wave function which represents a composite or summation of component waveforms having component frequencies ωᵢ ∈ [ω₁, ..., ωₙ], wherein n ≥ 2. In some variations of this embodiment, each of [ω₂, ..., ωₙ] is a resonant frequency of ω₁. Hence, in such variations, ωᵢ = αᵢω₁ for αᵢ ∈ Z (the set of integers).

It will be appreciated from the foregoing that, in some embodiments, the nested wave function may be expressed as the polynomial

$$Ω = \sum {}_{1}^{n} {a}_{k} {λ}_{k} ,$$

wherein λᵢ ∈ [λ₁, ..., λₙ] is a component waveform and αᵢ ∈ [α₁, ..., αₙ] is a real number coefficient which, in some applications, may be a weighting factor.

The component waveforms, if originating from the same source, are preferably in-phase. However, embodiments are also possible in which two or more of the component waveforms are out-of-phase by a predetermined amount to achieve various effects.

In other embodiments, brainwave entrainment may be accomplished through the use of composite waveforms which may be, for example, binaural waveforms having a periodically reoccurring succession of beats bᵢ ∈ [b₁, ..., bₙ], wherein n ≥ 2, wherein each beat bᵢ ∈ [b₁, ..., bₙ] is a binaural beat formed from corresponding component frequencies ωᵢ₁, ωᵢ₂, wherein each beat bᵢ ∈ [b₁, ..., bₙ] entrains at an effective frequency fᵢ ∈ [f₁, ..., fₙ], and wherein each of f₂, ..., fₙ is a resonance frequency of f₁. It will thus be appreciated that the resulting waveform Ω has as its domain the 2 x n frequency vector Ω = [ωᵢ₁, ωᵢ₂].

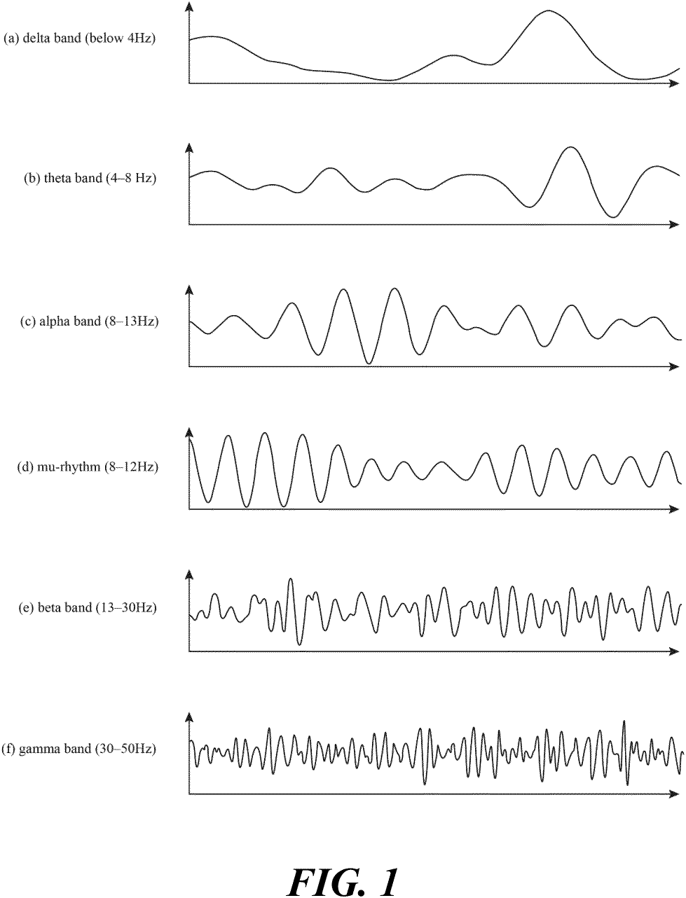

The composite waveforms described herein may utilize component waveforms of various frequencies and amplitudes, with the choice of frequency or amplitude depending, for example, on the characteristics desired for the composite waveform and the intended end use. By way of example but not limitation, the component waveforms may include waves selected from the group consisting of delta waves (0.5-4 Hz), theta waves (4-8 Hz), alpha waves (8-13 Hz), mu waves (also known as mu-rhythm waves) (8-12 Hz), beta waves (13-30 Hz) and/or the gamma waves (30-50 Hz). These waves are illustrated in FIG. 1.

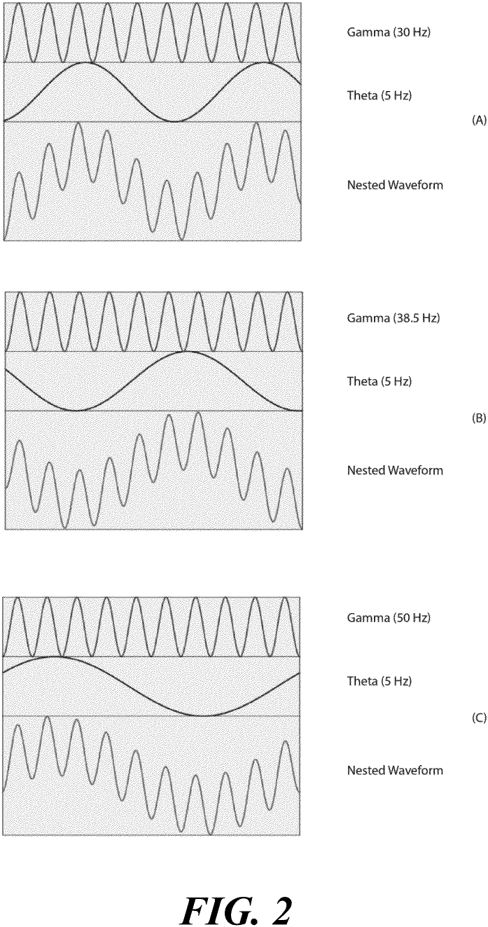

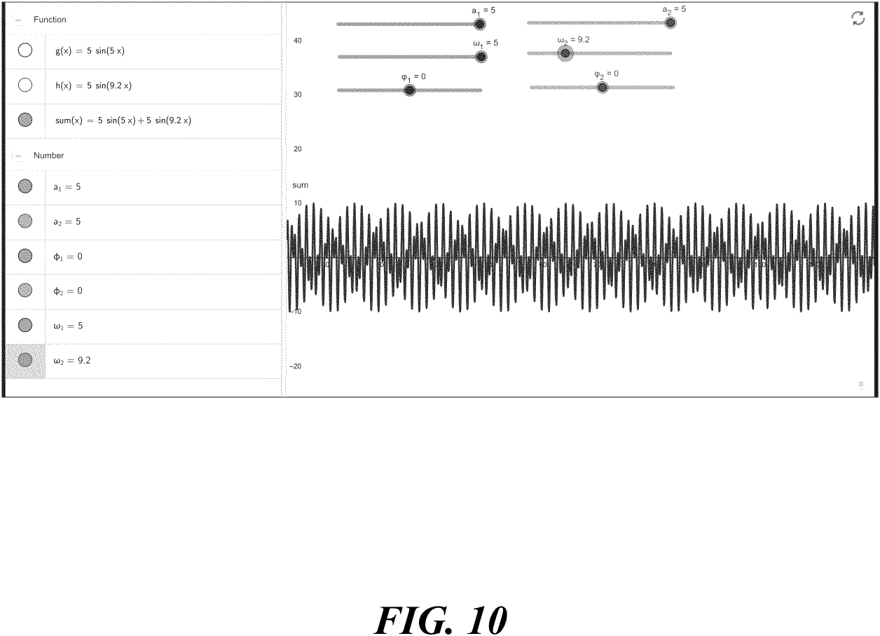

FIG. 2 illustrates a first particular, nonlimiting embodiment of a nested waveform which may be utilized in the systems and methodologies described herein. In the particular embodiment shown, the nested waveform is formed from first and second component wave forms. The component waves in this particular embodiment originate from the same source, have the same amplitude, and are in-phase. The first and second component waveforms are sine waves in this example. The first component waveform is a theta wave at 5 Hz, while the second component waveform is a gamma wave at 30 Hz (FIG. 2A), 38.5 Hz (FIG. 2B) or 50 Hz (FIG. 2C). Hence, in FIGS. 2A and 2C, the frequency of the gamma wave is an integer multiple (6 in FIGS. 2A, 10 in FIG. 2C) of the theta wave. In particular, in FIG. 2A, the gamma wave is the 6^{th} harmonic of the theta wave, while in FIG. 2C, the gamma wave is the 10^{th} harmonic of the theta wave. The gamma wave is not a harmonic of the theta wave in FIG. 2B.

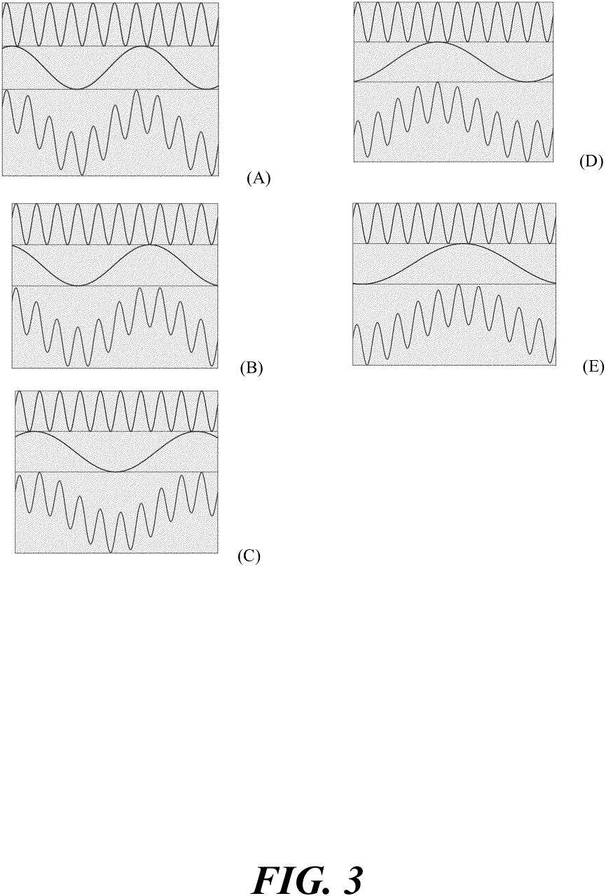

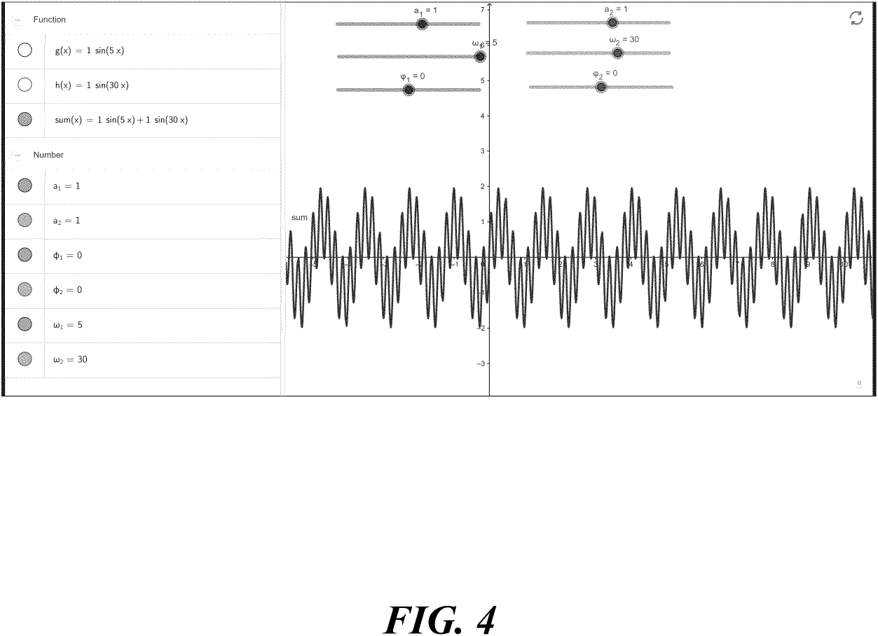

For reference, nested waveforms of a 5 Hz theta wave and gamma wave harmonics thereof are depicted in FIG. 3. Thus, FIG. 3A depicts the 6^{th} harmonic (30 Hz), FIG. 3B depicts the 7^{th} harmonic (35 Hz), FIG. 3C depicts the 8^{th} harmonic (40 Hz), FIG. 3D depicts the 9^{th} harmonic (45 Hz), and FIG. 3E depicts the 10^{th} harmonic (50 Hz). FIGS. 4-6 provide additional details for the waveforms of FIGS. 3A, 3C and 3E, respectively. The use of such harmonics may be preferred in certain applications of the devices and methodologies disclosed herein.

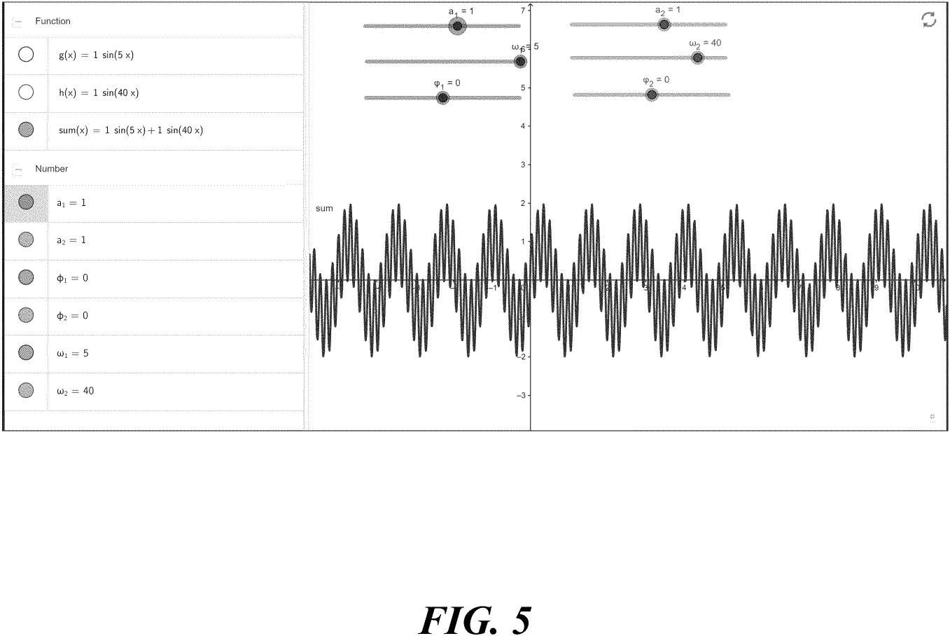

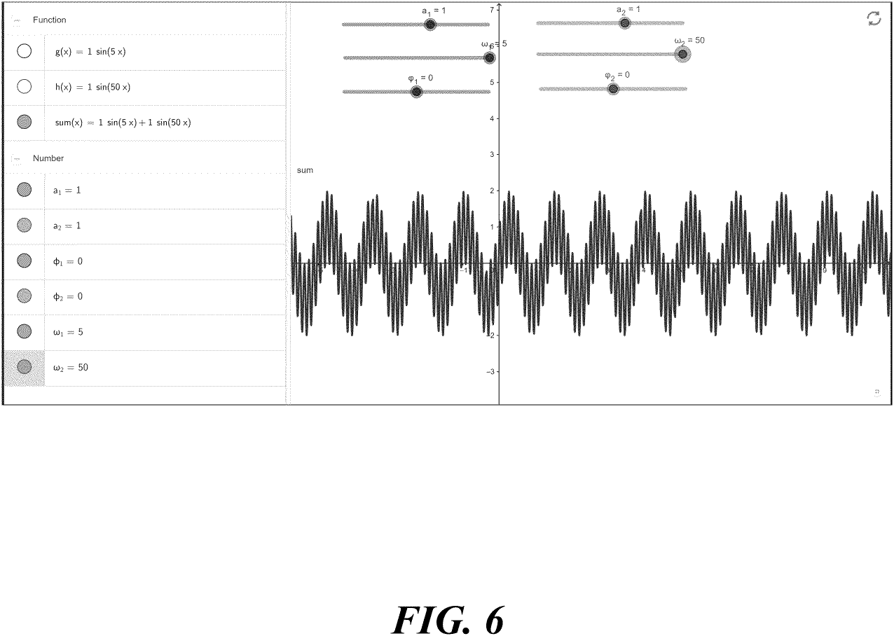

FIGS. 4-6 depict a series of nested waveforms created by combining a theta signal at 5 Hz with gamma signals of various frequencies. Thus, FIG. 4 depicts a nested waveform created by combining a theta signal at 5 Hz with a gamma signal at 30 Hz. FIG. 5 depicts a nested waveform created by combining a theta signal at 5 Hz with a gamma signal at 40 Hz. FIG. 6 depicts a nested waveform created by combining a theta signal at 5 Hz with a gamma signal at 50 Hz.

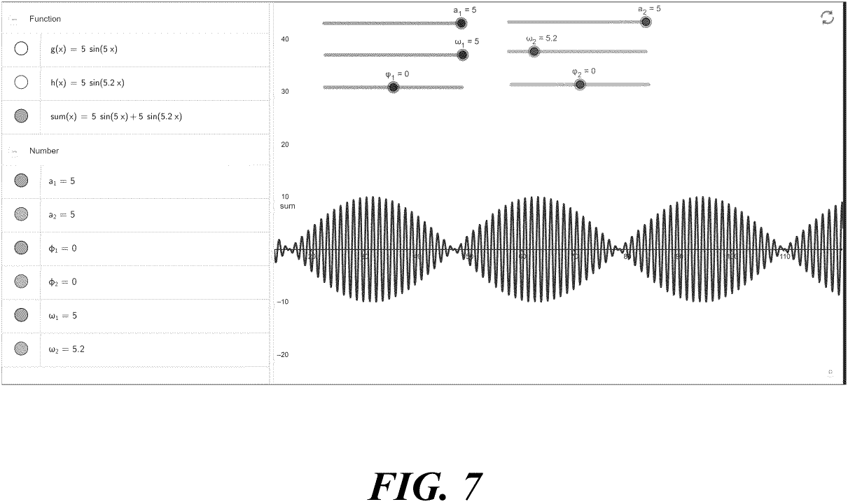

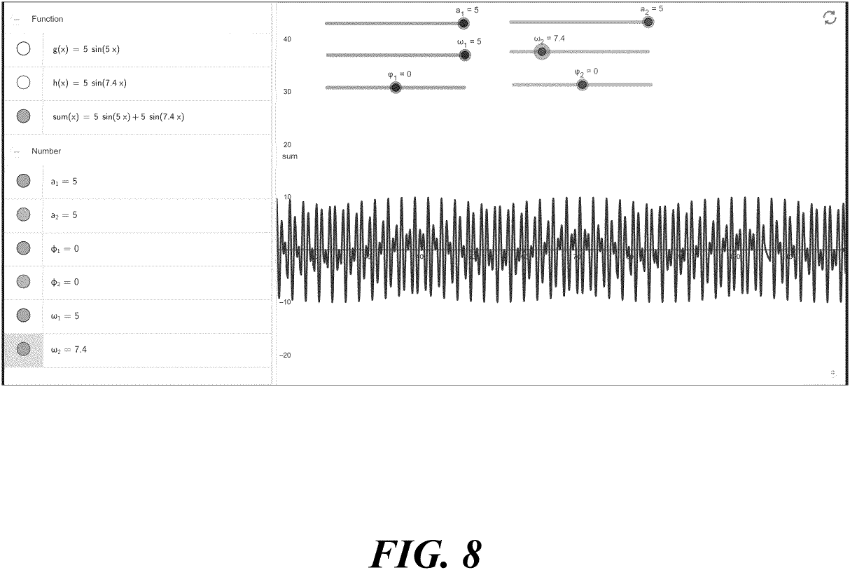

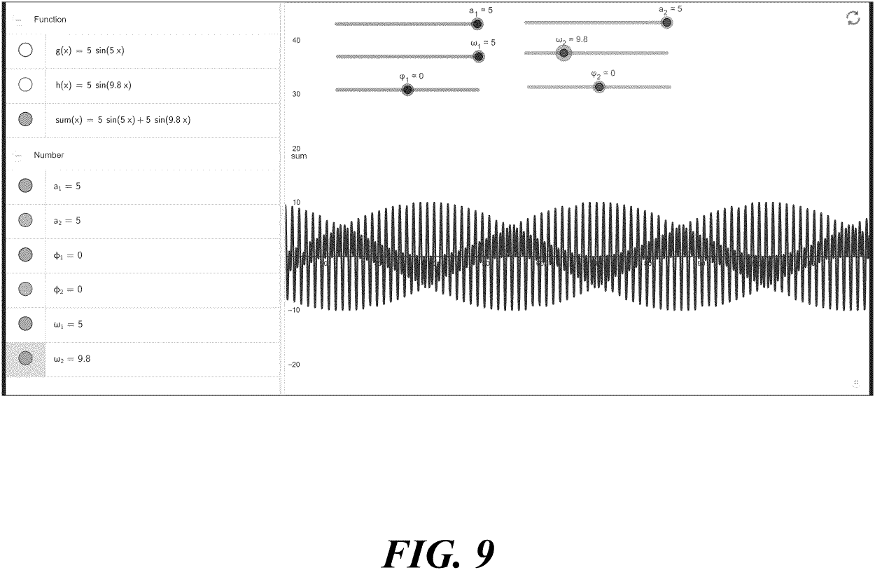

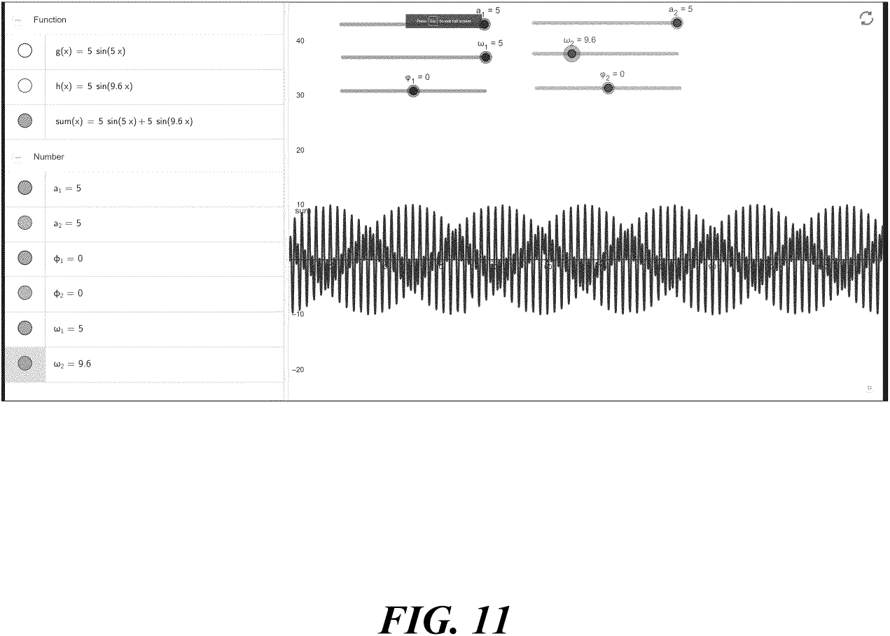

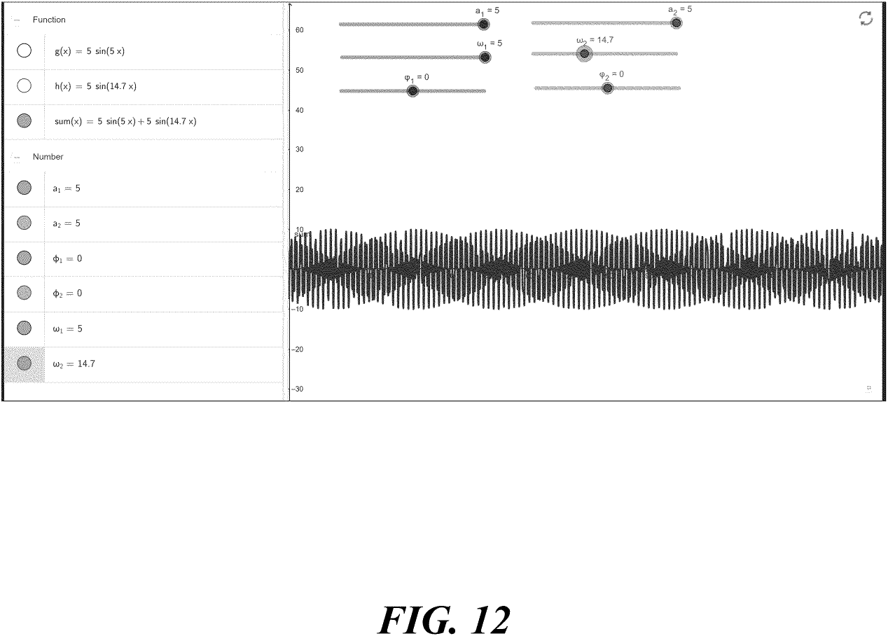

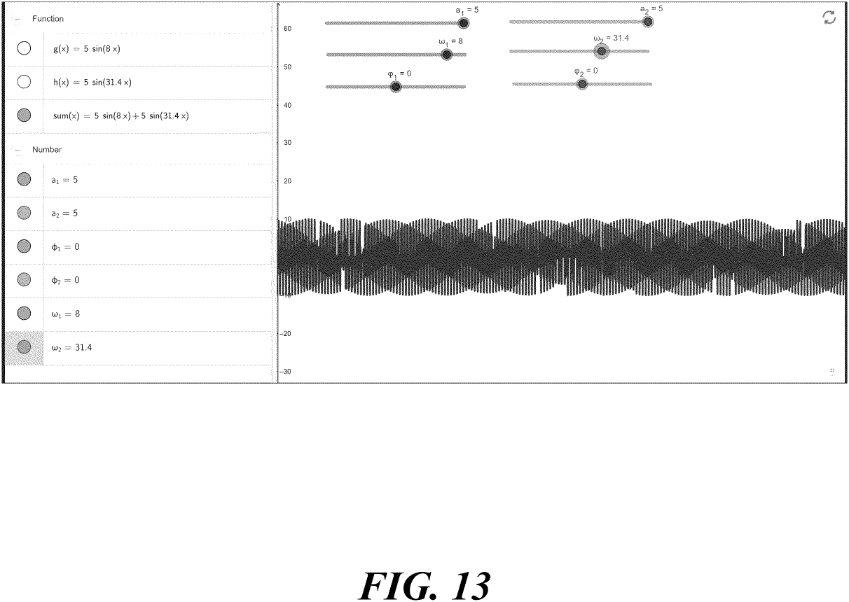

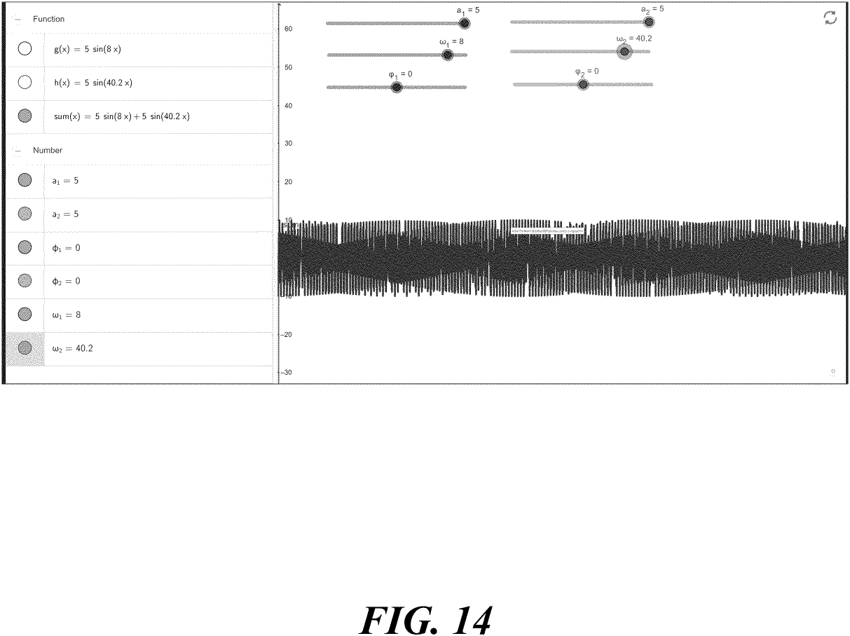

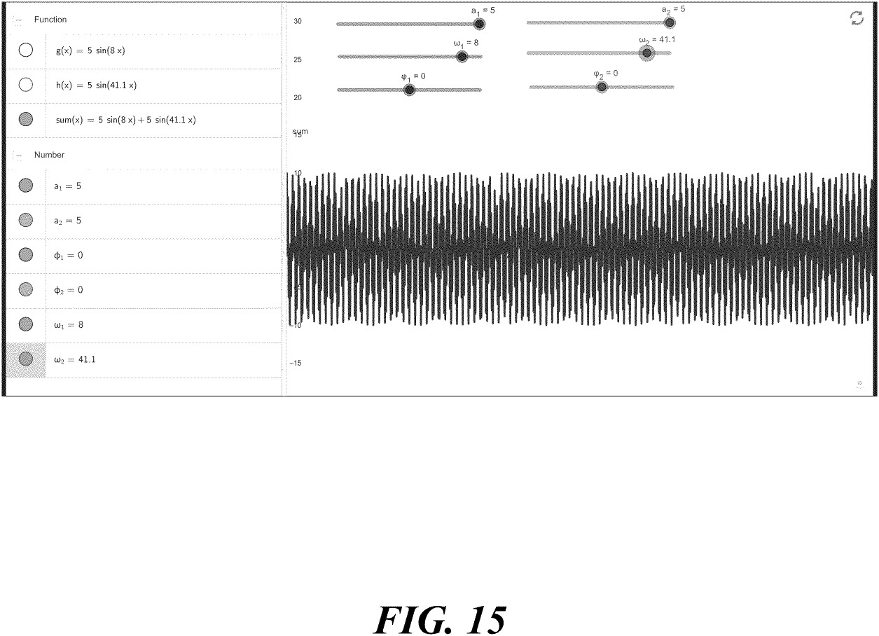

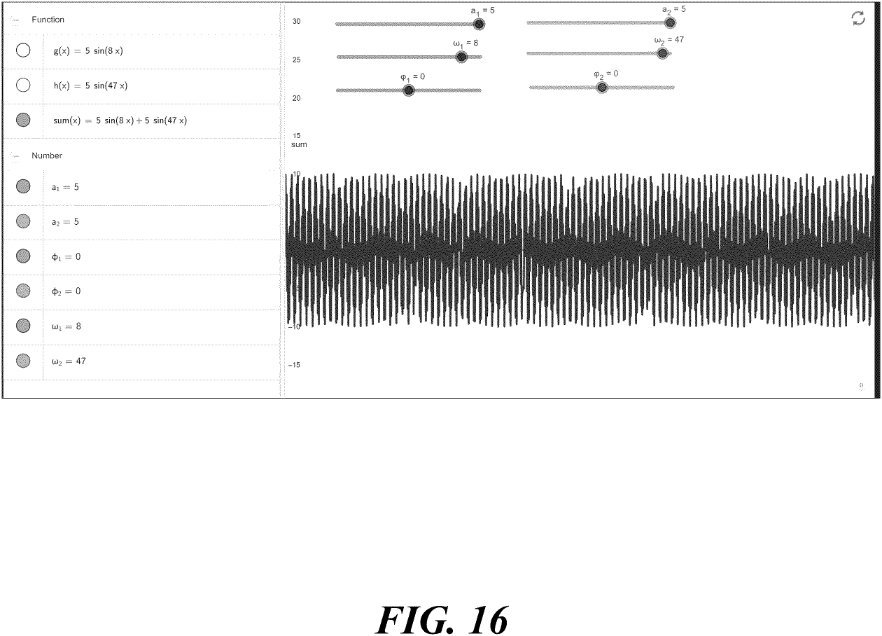

In some embodiments of the systems and methodologies described herein, two or more waveforms from a first frequency band may be utilized (for example, through summation) to form a composite waveform with a time-varying amplitude that oscillates in one or more other frequency bands. Several examples of such waveforms are depicted in FIGS. 7-16. For example, in FIG. 7, first and second theta waves having frequencies of 5 Hz and 5.2 Hz, respectively, are summed to generate a composite waveform whose amplitude oscillates at a frequency within the gamma region. FIG. 8 depicts a nested waveform made by combining theta signals of 5 Hz and 7.4 Hz to create a waveform having multiple periodic amplitude modulations. FIG. 9 depicts a nested waveform made by combining theta signals of 5 Hz and 9.8 Hz to create a waveform having an amplitude modulation of about 30 Hz. FIG. 10 depicts a nested waveform made by combining theta signals of 5 Hz and 9.2 Hz to create a waveform having an amplitude modulation of about 10 Hz. FIG. 11 depicts a nested waveform made by combining theta signals of 5 Hz and 9.6 Hz to create a waveform having an amplitude modulation of about 10 Hz. FIG. 12 depicts a nested waveform made by combining theta signals of 5 Hz and 14.7 Hz to create a waveform having multiple periodic amplitude modulations. FIG. 13 depicts a nested waveform made by combining theta signals of 8 Hz and 31.4 Hz to create a waveform having multiple periodic amplitude modulations. FIG. 14 depicts a nested waveform made by combining theta signals of 8 Hz and 40.2 Hz to create a waveform having multiple periodic amplitude modulations. FIG. 15 depicts a nested waveform made by combining theta signals of 8 Hz and 41.1 Hz to create a waveform having multiple periodic amplitude modulations. FIG. 16 depicts a nested waveform made by combining theta signals of 8 Hz and 47 Hz to create a waveform having multiple periodic amplitude modulations.

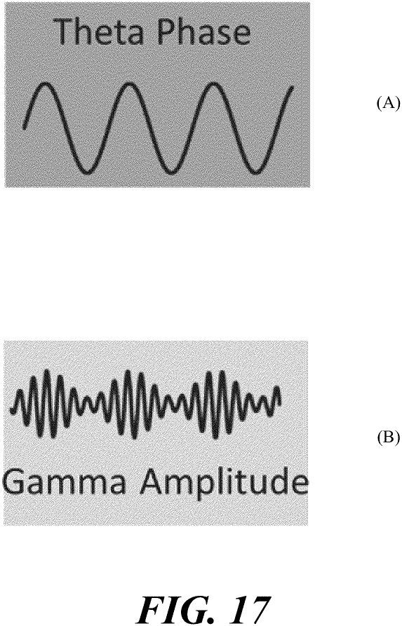

Some embodiments of the systems and methodologies described herein may utilize phase-amplitude coupling. Phase-amplitude coupling is a form of cross-frequency coupling where the amplitude of a high frequency signal is modulated by the phase of low frequency oscillations. This principle is depicted in FIG. 17, for the case in which the amplitude of a higher frequency gamma wave has been modulated with a lower frequency theta wave (FIG. 17A) to yield the waveform of FIG. 17B. Methods of forming such waveforms may utilize complex time frequency distributions such as, for example, the Reduced Interference Rihaczek (RID-Rihaczek) time-frequency distribution, to extract both the phase and amplitude components of the desired waveform within the frequency bands of interest. This approach is described, for example, in [Munia, T.T.K., Aviyente, S. Time-Frequency Based Phase-Amplitude Coupling Measure For Neuronal Oscillations. Sci Rep 9, 12441 (2019)], which is incorporated herein by reference in its entirety.

Other embodiments of the systems and methodologies described herein may utilize other forms of cross-frequency coupling. For example, in some embodiments, the phase of a low frequency signal may be modulated by the amplitude of high frequency oscillations (see, e.g., the non-limiting examples of FIGS. 2 and 3).

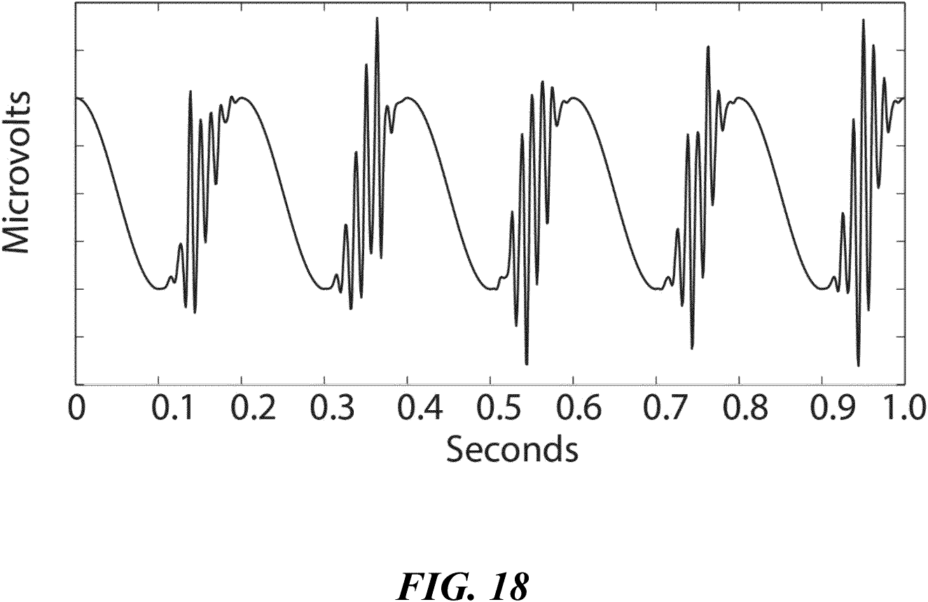

In some embodiments of the systems and methodologies disclosed herein, cross-frequency coupling between a plurality of oscillations may be implemented such that coupling occurs only during a portion of one of the oscillations. For example, FIG. 18 depicts an embodiment in which gamma oscillations (at 60 Hz) are phase-coupled to a theta oscillation (at 5 Hz) only during the rising phase of the theta oscillation. It will be appreciated that similar embodiments are possible in which the gamma oscillations are cross-frequency coupled to a theta oscillation only during the falling phase of the theta oscillation. It will also be appreciated that the gamma oscillations may be cross-frequency coupled to the theta oscillation during any arbitrary portion (or portions) of the time domain of the theta oscillation.

Moreover, while the foregoing discussion has been limited to gamma and theta waves for purposes of simplicity, the same principle may be applied to produce waveforms based on cross-frequency coupling using waves selected from various distinct bands including, for example, any of the bands depicted in FIG. 1. For example, consider the set of n waveforms f₁(t), ...., fₙ(t) having associated frequencies ω₁, ...., ωₙ, wherein ω₁ < .... < ωₙ, and wherein n ≥ 2. Embodiments are possible in accordance with the teachings herein in which oscillations f₂(t), ...., fₙ(t) are cross-frequency coupled to the oscillation f₁(t) in various ways.

For example, in some embodiments, oscillations f₂(t), ...., fₙ(t) may be cross-frequency coupled to the oscillation f₁(t) in a continuous manner. FIG. 2 provides an example of this type of embodiment in which gamma oscillations at 30 Hz (FIG. 2A), 38.5 Hz (FIG. 2B) and 50 Hz (FIG. 2C), respectively, are cross-frequency coupled to a theta oscillation of 5 Hz. In the particular, non-limiting embodiment of FIG. 2, the gamma and theta oscillations happen to have the same amplitude. However, various embodiments are possible in which the cross-frequency coupled oscillations have different amplitudes.

Embodiments are also possible in which oscillation f₁(t) is subjected to cross-frequency coupling in a time-varying manner. FIG. 4 depicts a particular, non-limiting embodiment of this type of embodiment. In the waveform depicted therein, a theta oscillation of 5 Hz is successively cross-frequency coupled with gamma oscillations of 30 Hz, 40 Hz and 50 Hz over respective time intervals t₁, ..., t₅ as depicted in FIGS. 4-6, respectively. It will be appreciated that a composite waveform may be constructed by combining these nested waveforms in various sequences. For example, if a series of nested oscillations φ₁ (t), ...., φₙ(t) having corresponding higher frequency components [ω₁, ..., ωₙ] is produced in the foregoing manner, then a composite waveform may be constructed in which each period of the waveform is a nested oscillation φᵢ ∈ [φ₁ (t), ...., φₙ(t)]. In some cases, the resulting waveform may feature successive periods or segments in which the frequency of the higher component is monotonically increasing or decreasing. Thus, for example, if ω₁ < ··· < ωₙ, then the composite waveform may have a sequence of consecutive periods or segments with associated frequencies of the higher frequency component of ω₁, ..., ωₙ. The composite waveform may also have a sequence of consecutive periods or segments with associated frequencies of the higher frequency component of ωₙ, ..., ω₁, or of ω₁, ..., ωₙ, ωₙ, ..., ω₁.

Embodiments are also possible in which multiple oscillations within one frequency band can be combined to obtain a waveform with features that oscillate in another frequency band. This may include phase-amplitude coupling (PAC), a form of cross-frequency coupling where the amplitude of a high frequency signal is modulated by the phase of low frequency oscillations.

While the use of sinusoidal or geometric waveforms is preferred in the devices and methodologies described herein, various other types of waveforms may be utilized in the devices and methodologies disclosed herein. These include, without limitation, triangle waveforms, square waveforms, sawtooth waveforms, or combinations of the foregoing. Of course, it will be appreciated that the latter waveforms can be expressed to any desired degree of accuracy as a summation of sine waves. In addition, the amplitudes and/or frequencies of these waveforms may be time-varying or frequency-varying.

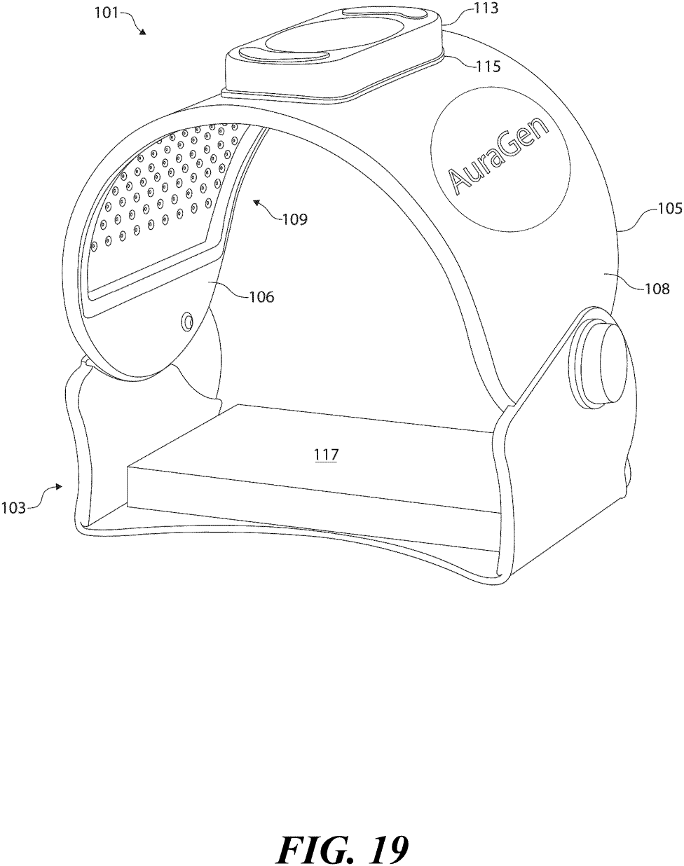

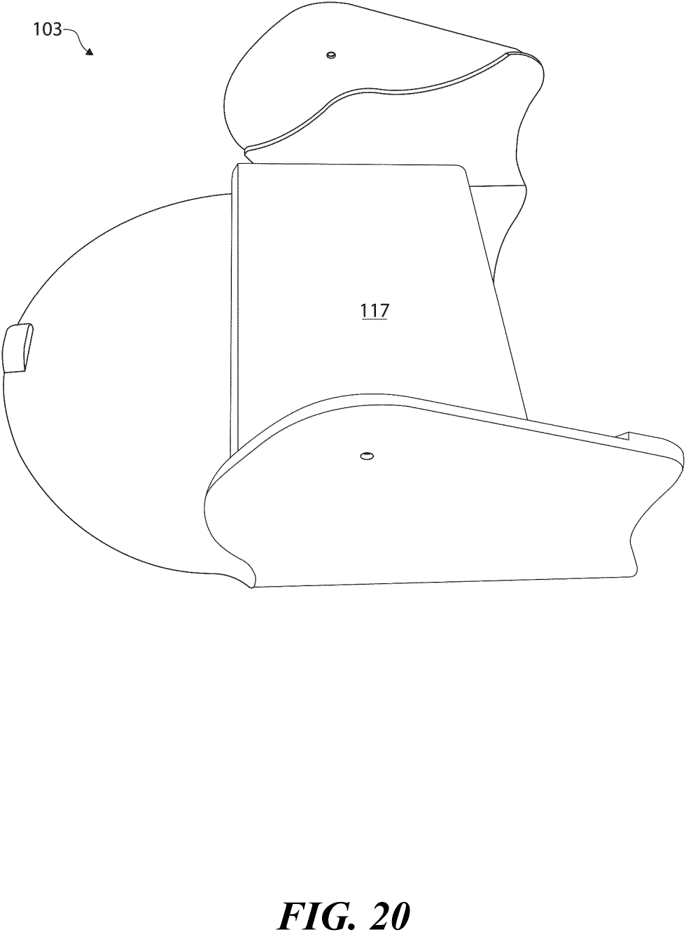

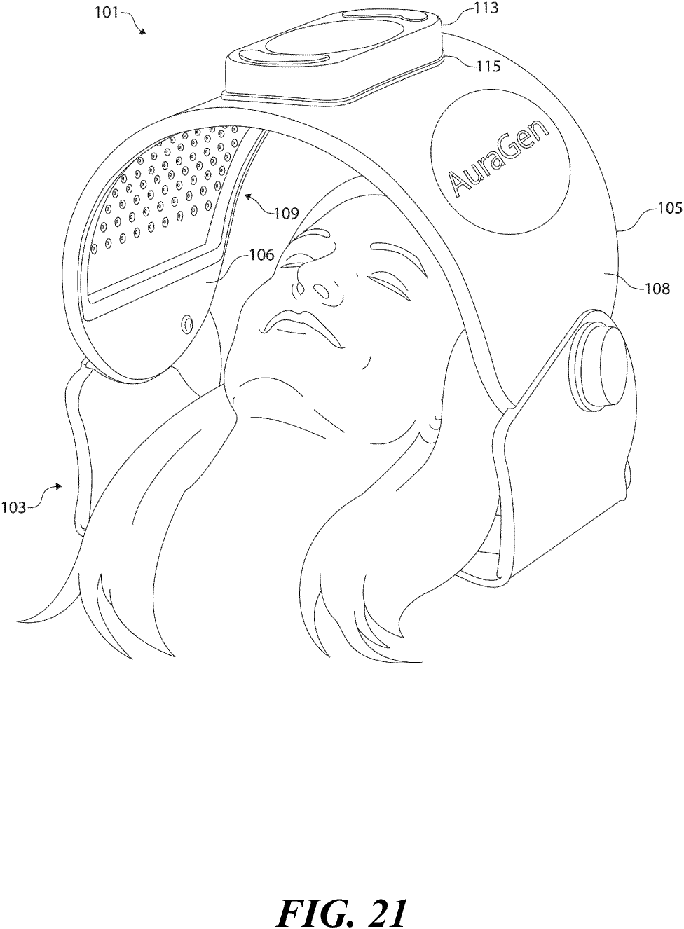

Various devices may be utilized to implement open-loop brainwave entrainment in accordance with the teachings herein, and these devices may utilize any of the waveforms described above. FIGS. 19-22 illustrate a particular, nonlimiting embodiment of such a device. The entrainment device 101 depicted therein comprises a base 103 (shown in isolation in FIG. 20) having a peripheral element 105 attached thereto and, optionally, an audio headset (not shown; the need for a headset may be determined, for example, by whether the entrainment methodology uses traveling waves originating from the same source, or standing waves generated by two distinct sources). The base 103 and peripheral element 105 define an opening 107 in which a user’s head is placed (see FIG. 21). The base 103 and/or peripheral element 105 may be equipped with an audio jack, a Bluetooth transmitter, or other suitable provisions as necessary or desirable to support the use of an audio headset by the user.

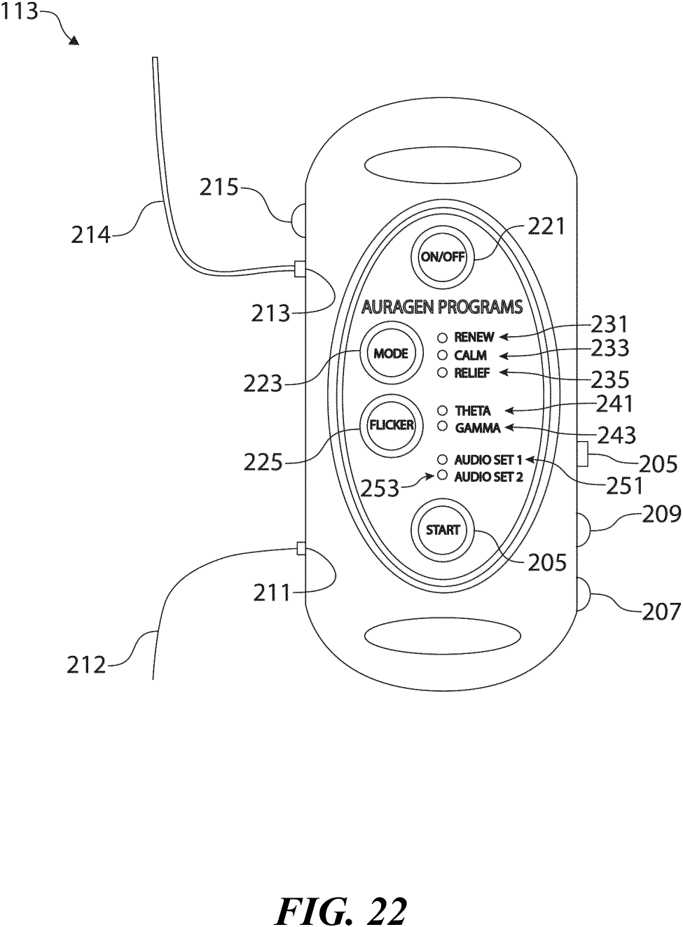

The base 103 in this particular embodiment is equipped with a pillow 111 for user comfort, and to provide the user with the ability to lie down or sleep during a brainwave entrainment session. The peripheral element 105 has a first major inward-facing surface 106 and a second major outward-facing surface 108. The first major surface 106 is equipped with an LED array 109 which can be activated with a remote control 113 to illuminate the user’s head at one or more wavelengths. The second major surface 108 is equipped with a holder 115 for the remote control 113. The remote control 113, which is shown in greater detail in FIG. 22, may also be utilized to modulate the light emitted by the LED array 109, to select one or more wavelengths of light emitted by the LED array 109, and to control the playback of one or more audio files or tracks.

In use, a user’s head is placed in the opening 107 such that the back of the user’s head is on the pillow 111 and such that the user is facing the first major surface 106 of the peripheral portion 105 as shown in FIG. 21. The user (or possibly a clinician or other assistant) then uses the remote control 113 to activate the entrainment device 101 and to cause it to function in one or more selected modes. Regarding the latter, it is to be noted that the entrainment device 101 may be programmed with various algorithms which cause it to function in particular ways, some of which are described in greater detail below. The entrainment device 101 may also be programmed to play music or soundtracks, which may be advantageously matched to the particular algorithm being implemented by the entrainment device 101.

In some embodiments, the entrainment device may include a port to allow plugin of additional LED portable devices that may be place in the mouth of the user (via, for example, a mouth guard). In other embodiments, the entrainment device may include a small pad that may be wrapped or directly applied to a specific body part of the user. In still other embodiments, the entrainment device may include a set of googles or glasses that are placed over the eyes of the user to provide focused treatment to those areas, or to prevent treatment of those areas. Of course, it will be appreciated that any of the foregoing accessories may be utilized in combination in various embodiments of the systems and methodologies disclosed herein.

Various LEDs 109 or other light sources which emit at various wavelengths may be utilized in the devices and methodologies disclosed herein. However, the use of light sources which emit at wavelengths in the red, infra-red and blue-turquoise regions of the spectrum (as shown in FIGS. 5, 6 and 7, respectively) are preferred, and the use of light sources which emit at about 470 nm, 670 nm and 870 nm are especially preferred. In a preferred mode of operation, these light sources are made to oscillate or flicker in the theta or gamma band.

It will be appreciated that light may be emitted at the foregoing wavelengths in various manners, including sequentially or simultaneously. For example, the LED array 109 may be operated to emit electromagnetic radiation at a single wavelength (i.e., monochromatically) or at multiple wavelengths. In some cases, the LED array 109 may include a first set of LEDs that are operated to emit light at a first wavelength, a second set of LEDs that are operated to emit light at a second wavelength, and (optionally) a third set of LEDs that are operated to emit light at a third wavelength. In other cases, the LED array 109 may be operated such that all of the LEDs in the array emit light at a first wavelength for a first period of time, all of the LEDs in the array emit light at a second wavelength for a second period of time, and (optionally) all of the LEDs in the array emit light at a third wavelength for a third period of time.

The particular wavelength(s) of emission of the LED array 109, the duration of those emissions, the frequency of oscillation (if any), the intensity of the emitted light, the selection of accompanying audio tracks or files (if any), and/or the oscillation of any accompanying audio tracks, files or component(s) thereof, may be selected to achieve a desired physiological or psychological effect. It will be appreciated that, in some embodiments, the duration of emission for any particular wavelength of light may remain constant or may vary during the course of a therapy session. It will further be appreciated that, in some embodiments, any of the LEDs in the LED array 109 may be operated to emit two or mor wavelengths of light, including broadband radiation or white light.

The brainwave entrainment devices and methodologies disclosed herein may be utilized as an effective tool in treating a subject for certain psychological or physiological conditions, or for prevention of these conditions. These conditions include, but are not limited to, traumatic brain injury, addiction or dependence (including, for example, addiction to, or dependence on, opioids, amphetamines, stimulants, alcohol or cannabis), depression (and more specifically, clinical depression or major depression), PTSD, developmental trauma disorder, traumatic brain injury and its sequelae, and Alzheimer’s disease. In a preferred embodiment of the methodology disclosed herein, a subject is first diagnosed as suffering from one of the foregoing conditions, and then brainwave entrainment is utilized to treat the subject.

Various aspects of the systems and methodologies described herein have been described above with respect to the particular, non-limiting embodiments disclosed herein. It will be appreciated that these various aspects may be employed in various combinations (including various sub-combinations) or permutations in accordance with the teachings herein.

For example, while the use of light sources which emit at wavelengths in the red, infra-red and blue-turquoise regions of the spectrum are preferred, and the use of light sources which emit at about 470 nm, 670 nm and 870 nm are especially preferred, it will be appreciated that the devices and methodologies disclosed herein may utilize various other frequencies or wavelengths of electromagnetic radiation to achieve desired physiological or psychological effects. These wavelengths or frequencies may be selected, for example, from the visible, infrared or ultraviolet regions of the electromagnetic spectrum.

Similarly, in a preferred mode of operation, the intensities of one or more of these light sources are made to oscillate or flicker in the theta or gamma frequency band during at least a portion of a therapy session. However, embodiments are possible in which the light sources are made to oscillate or flicker at other frequencies, or in which the light sources (or elements thereof) operate in a manner which is not time varying. Embodiments are also possible in which the light sources are made to oscillate or flicker at harmonics of the foregoing frequencies.

While the embodiment of FIGS. 19-22 is a preferred embodiment of the brainwave entrainment device described herein, it will be appreciated that brainwave entrainment devices of various shapes, configurations, layouts and functionalities may be utilized in the practice of the methodologies disclosed herein, and these light therapy units may be provided with various accessories.

For example, in some embodiments, brainwave entrainment devices may be utilized that are adapted to illuminate one or more inner surfaces of a subject’s oral cavity. In such embodiments, a light therapy unit utilized for this purpose may be fashioned as a standalone device, while in other embodiments, such a light therapy unit may be fashioned as an accessory to a main light therapy unit which is utilized to illuminate the outer surfaces of a subject’s head. In embodiments of the latter type, the accessory may be adapted to communicate with the main brainwave entrainment device such that the accessory is controlled by, or acts in concert with, the main brainwave entrainment device.

In some instances of embodiments of a brainwave entrainment device adapted to illuminate one or more inner surfaces of a subject’s oral cavity, the light therapy unit may be equipped with a mouth guard which is in optical communication with a light source by way of a suitable light guide, and which distributes light received from the light source in a suitable manner. In some cases, the mouthguard may be customized to the user. By way of example but not limitation, such a mouth guard may be adapted to direct suitable wavelengths of light to various surfaces of the oral cavity of a subject, including the teeth, gums, upper or lower mouth, and throat. The mouth guard, light guide or portions thereof may be equipped with suitable materials that specularly or diffusely transmit or reflect incident radiation in one or more directions. In addition to their possible use in treating physiological or psychological conditions, these embodiments may offer additional benefits such as, for example, the treatment or prevention of gingivitis and other bacterial infections.

In some embodiments of the devices disclosed herein, measures may be taken to ensure that the brainwave entrainment device is applied to only specific parts of the user’s body. For example, in some embodiments, the aforementioned light therapy unit which is adapted to illuminate one or more inner surfaces of a subject’s oral cavity may be used by itself such that only these surfaces are exposed to the brainwave entrainment therapy. Similarly, in some embodiments, the user may be equipped with glasses or goggles such that the user’s eyes or optical nerves are not exposed to the brainwave entrainment light, or such that this light is concentrated on the user’s eyes or optical nerves. In still other embodiments, an optical pad or other suitable means may be utilized to apply brainwave entrainment device only to the back of a user’s neck, or to a user’s chest (alone or in combination with the application of entraining frequencies to the user’s head).

Preferred embodiments of the devices disclosed herein are adapted to allow the user to lie down or otherwise assume a state of repose during a brainwave entrainment session. Such embodiments may include, for example, a pillow or one or more deformable pads which support the user’s head during brainwave entrainment therapy. Here, it is notable that many other devices in the art which are designed for brainwave entrainment therapy require the user to remain in a sitting or standing position for the duration of the therapy.

In some embodiments of the devices disclosed herein, the device may be equipped with a suitable controller, which may be wireless or wired. The controller may be programmable or pre-programmed, and may be equipped with suitable programming instructions (which may include an operating system) recorded in a tangible, non-transient medium that cause the brainwave entrainment device to operate in various modes or to perform various functions. These modes or functions may be selected or optimized for the treatment of various portions of a subject’s body, or for the treatment of particular physiological or psychological conditions.

Various parameters (and ranges of these parameters) may be utilized in the brainwave entrainment devices and methodologies disclosed herein. These include, without limitation, wavelength, frequency, entrainment waveform, energy, fluence, power, irradiance, intensity, pulse mode, treatment duration, and repetition. These parameters and their values may be selected to treat a subject for certain psychological or physiological conditions, to lessening the severity or effects of these conditions, and/or to preventing the occurrence of these conditions. These conditions include, but are not limited to, traumatic brain injury, opioid addiction (including, for example, heroin addiction or addiction to prescription opioids), depression (and more specifically, clinical depression or major depression), mild cognitive impairment, dementia, Alzheimer’s disease and developmental trauma.

It will be appreciated that the brainwave entrainment devices disclosed herein, and the components thereof, may be equipped with suitable optical elements to achieve various purposes. Such optical elements (or portions thereof) may be diffusely or specularly reflective or transmissive. Suitable optical elements may include, but are not limited to, reflective elements, polarizers, color shifting elements, filters, light guides (including, without limitation, optical fibers, light pipes and waveguides), prismatic elements, lenses (including Fresnel lenses), and lens arrays.

In preferred embodiments of the systems and methodologies disclosed herein, one or more audio tracks or audio files may be provided that may be modulated, coordinated and/or synchronized with the plurality of LEDs or the light emitted therefrom. Preferably, the audio tracks or audio files include sound that is modulated, coordinated and/or synchronized with the LEDs or the light emitted therefrom at one or more frequencies selected from the ranges depicted in FIG. 1. The audio tracks or files (alone, or in combination with any light wavelengths utilized) may be selected to achieve a desired physiological or psychological effect in the user, either alone or in combination with the light therapy.

One skilled in the art will further appreciate that the systems and methodologies disclosed herein may be used not only to treat various physiological or psychological conditions, but to prevent them from occurring in the first place. For example, these systems and methodologies may be adapted to prevent the onset of depression, PTSD, ADHD, opioid addiction (for example, heroine or oxycodone), or conditions resulting from traumatic brain injury, or of conditions which might otherwise result from the foregoing.

The systems and methodologies disclosed herein may be utilized in conjunction with other methodologies or techniques. For example, these systems and methodologies may be used in combination with emotional freedom technique (EFT) tapping. EFT tapping is a holistic healing technique that may be utilized to treat various issues including, without limitation, stress, anxiety, phobias, emotional disorders, chronic pain, addiction, weight control, and limiting beliefs. EFT tapping involves tapping with the fingertips on specific meridian endpoints of the body, while focusing on negative emotions or physical sensations. Proponents of the method claim that it calms the nervous system, rewires the brain to respond in healthier ways, and restores the body’s balance of energy.

One skilled in the art will further appreciate that the optimal parameters for a brainwave entrainment session may depend on a variety of factors including, but not limited to, the condition being treated (or prevented), the physiological or psychological state of the user, the user’s biometrics, and other such factors. In some use cases, selection of these parameters may be made by, or in coordination with, a physician, a psychiatrist, or other healthcare provider. These parameters may include, but are not limited to, the wavelengths of light to be utilized, the audio tracks or files to accompany the light therapy, the frequencies of oscillation utilized for the intensity in any of the wavelengths or light or sound, the portions of the user’s head or body to be exposed to the light therapy, and the duration of the treatment.

While the devices and methodologies disclosed herein have frequently been described with reference to the use of traveling waves originating from a common source, one skilled in the art will appreciate that various embodiments of these methodologies and devices may also be produced which utilize waves originating from distinct sources (e.g., standing waves). In some embodiments, various devices, materials or other such measures may be taken to cause or prevent reflection of the waves used for brainwave entrainment.

The above description of the present invention is illustrative and is not intended to be limiting. It will thus be appreciated that various additions, substitutions and modifications may be made to the above described embodiments without departing from the scope of the present invention. Accordingly, the scope of the present invention should be construed in reference to the appended claims. It will also be appreciated that the various features set forth in the claims may be presented in various combinations and sub-combinations in future claims without departing from the scope of the invention. In particular, the present disclosure expressly contemplates any such combination or sub-combination that is not known to the prior art, as if such combinations or sub-combinations were expressly written out.

What is claimed is:

1. A method for performing brainwave entrainment on a subject, comprising:
providing a signal source selected from the group consisting of audio signal sources and video signal sources; and
using the signal source to apply to the subject, or to induce the formation of in the subject, at least one nested waveform selected from the group consisting of audio waves and electromagnetic waves.

2. The method of claim 1, wherein said brainwave entrainment is open-loop audio visual stimulation (AVS) brainwave entrainment.

3. The method of claim 1, further comprising:
providing an audio-visual stimulation (AVS) device; and
using the AVS device to apply to the subject, or to induce the formation in the brain of the subject, at least one nested waveform selected from the group consisting of audio waves and electromagnetic waves.

4. The method of claim 1, wherein the nested waveform is a theta-nested gamma waveform.

5. The method of claim 1, wherein the nested waveform is a composite waveform of a first waveform having a first frequency, and a second waveform having a second frequency.

6. The method of claim 5, wherein the first and second waveforms are selected from the group consisting of delta waves, theta waves, alpha waves, beta waves and gamma waves.

7. The method of claim 5, wherein the first frequency is within the range of 0.5 Hz to 2 Hz.

8. The method of claim 5, wherein the first frequency is within the range of 4 Hz to 8 Hz.

9. The method of claim 5, wherein the first frequency is within the range of 8 Hz to 12 Hz.

10. The method of claim 5, wherein the first frequency is within the range of 12 Hz to 38 Hz.

11. The method of claim 5, wherein the first frequency is within the range of 12.5 Hz to 30 Hz.

12. The method of claim 5, wherein the first frequency is within the range of 25 Hz to 140 Hz.

13. The method of claim 5, wherein the first frequency is within the range of 38 Hz to 42 Hz.

14. The method of claim, wherein the second waveform is a higher harmonic of the first waveform.

15. The method of claim 1, wherein the nested waveform is a composite waveform of a first waveform having a first frequency, a second waveform having a second frequency and a third waveform having a third frequency.

16. The method of claim 15, wherein the first, second and third waveforms are selected from the group consisting of delta waves, theta waves, alpha waves, beta waves and gamma waves.

17. The method of claim 16, wherein the third waveform is a higher harmonic of the first waveform.

18. The method of claim 15, wherein the second and third waveforms are higher harmonics of the first waveform.

19. The method of claim 1, wherein the nested waveform is a composite waveform of a first waveform having a first frequency, a second waveform having a second frequency, a third waveform having a third frequency, and a fourth waveform having a fourth frequency.

20. The method of claim 19, wherein the first, second, third and fourth waveforms are selected from the group consisting of delta waves, theta waves, alpha waves, beta waves and gamma waves.

21. The method of claim 20, wherein the nested waveform is a composite waveform of a first waveform having a first frequency, a second waveform having a second frequency, a third waveform having a third frequency, a fourth waveform having a fourth frequency and a fifth waveform having a fifth frequency.

22. The method of claim 20, wherein the first, second, third, fourth and fifth waveforms are selected from the group consisting of delta waves, theta waves, alpha waves, beta waves and gamma waves.

23. The method of claim 1, wherein the nested waveform is a composite of a first wave selected from a first frequency band, and a second wave selected from a second frequency band which is distinct from the first frequency band, and wherein the first and second frequency bands are selected from the group consisting of the delta band, theta band, alpha band, mu-rhythm band, beta band and gamma band.

24. The method of claim 23, wherein the nested band is formed by cross-frequency coupling of the first and second waves.

25. The method of claim 23, wherein the nested band is formed by phase coupling of the first and second waves.

26. The method of claim 23, wherein the nested band is formed by phase-amplitude coupling of the first and second waves.

27. The method of claim 23, wherein the second wave is a harmonic of the first wave.

28. The method of claim 1, wherein the signal source is used to apply to the subject at least one nested waveform selected from the group consisting of audio waves and electromagnetic waves.

29. The method of claim 1, wherein the signal source is used to apply to induce the formation of in the subject at least one nested waveform selected from the group consisting of audio waves and electromagnetic waves.

30. The method of claims 28-29, wherein the at least one nested waveform is an audio waveform.

31. The method of claims 28-29, wherein the at least one nested waveform is an electromagnetic waveform.

32. A method for treating an individual who has, or is at risk of developing, a psychological disorder, comprising:
determining that an individual has, or is at risk of developing, a psychological disorder; and
performing brainwave entrainment on the individual using any of the methods of claims 1-31.
